(19) United States
(12) Patent Application Publication
(10) Pub. No.: US 2005/0004604 A1
(43) Pub. Date: Jan. 6, 2005

(54) Artificial larynx using coherent processing to remove stimulus artifacts

(76) Inventor: Jerry Liebler, North Plains, OR (US)

Correspondence Address: GIFFORD, KRASS, GROH, SPRINKLE;ANDERSON & CITKOWSKI, PC, 280 N OLD WOODARD AVE, SUITE 400, BIRMINGHAM, MI, 48009, US

(21) Appl. No.: 10/861,960

(22) Filed: Jun. 4, 2004

Related U.S. Application Data
(62) Division of application No. 10/021,192, filed on Oct. 30, 2001, now Pat. No. 6,698,394.
(63) Continuation-in-part of application No. 09/937,543, filed on Sep. 26, 2001, now abandoned.
(60) Provisional application No. 60/244,349, filed on Oct. 30, 2000.
(60) Provisional application No. 60/252,280, filed on Nov. 21, 2000.
(60) Provisional application No. 60/260,256, filed on Jan. 8, 2001.
(60) Provisional application No. 60/267,598, filed on Feb. 9, 2001.
(60) Provisional application No. 60/125,798, filed on Mar. 23, 1999.
(60) Provisional application No. 60/134,457, filed on May 17, 1999.
(60) Provisional application No. 60/141,166, filed on Jun. 25, 1999.
(60) Provisional application No. 60/147,584, filed on Aug. 6, 1999.

(51) Int. Cl. A61N 1/18

(52) U.S. Cl. 607/2

(57) ABSTRACT

An artificial larynx processes a microphone's output coherently with the stimulus used to excite the vocal cavities. The use of coherent processing, implemented with a matched filter or a comb filter, allows complete removal of all of the stimulus from the recovered audio for a much cleaner reproduction. The coherent processing is preferably carried out in a digital signal processor (DSP) interfaced to an audio analog-to-digital (A/D) converter and other circuitry, including digital-to-analog converters (DACs). A microphone feeds the A/D, while the DACs feed amplifiers driving loudspeakers.

### FIELD OF THE INVENTION

This invention related to prosthetic larynx devices and, in particular, to such a device that uses coherent processing to remove unwanted acoustic artifacts resulting from the stimulus used.

### BACKGROUND OF THE INVENTION

When most people speak, they produce a base sound or “pitch” with their vocal chords. This base sound is then modified by changing the shape and size of oral or nasal structures to form words and sentences. When a person has a larangectomy, due to disease or trauma, the mechanism to produce the pitch is removed. As a result, speech is not possible without some form of prosthetic device.

The first prosthetic devices were vibrators that were held to the throat and turned on by pushbuttons when speech was desired. These devices masked much of the speech by the vibrator's output. Other prosthetic devices use transducers located inside the mouth (inter-oral) to reduce the amount of stimulus heard by the listener.

Over the years, more sophisticated electronic solutions have become available. One example is disclosed in U.S. Pat. No. 5,828,758, the entire content of which is incorporated herein by reference. This patent describes a system for monitoring a user's oral-nasal cavity including a sound source, a sensor, and a circuit. The sound source provides a first signal in the cavity. The sensor receives a second signal modulated by the cavity. The second signal is affected in part by the first signal and in part by the cavity. The sensor provides a monitor signal having a first modulation and a first period. The circuit, which is coupled to the sensor, determines a third signal. The third signal includes a second modulation responsive to the first modulation and includes a second period unequal to the first period.

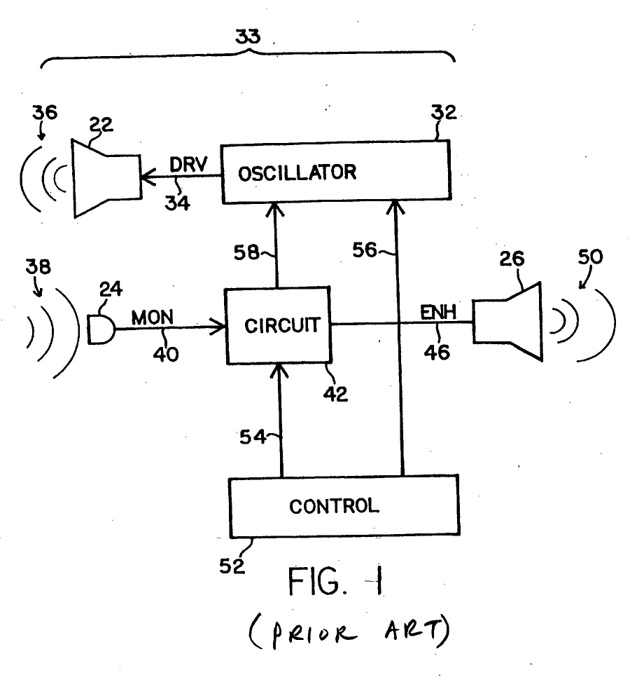

FIG. 1 is a block diagram of an embodiment taken from the '758 patent. Oscillator 32 generates drive signal DRV on line 34 to transducer 22. Transducer 22 emits sound signal 36 which is directed toward the user's oral-nasal cavity. The cavity re-radiates sound signal 38 which includes part of the spectral energy of sound signal 36 as amplified and attenuated by the nonlinearities and resonances of the cavity. The distribution of spectral energy in signal 38 is called a modulation, and includes the spectral energy of the user's voice and consonant sounds, if any. As the user moves his or her mouth, tongue, teeth, and lips, the nonlinear and resonant characteristics of the cavity change. Therefore, the modulation of sound signal 38 conveys information about the cavity with or without the user's voice.

Oscillator 32 and transducer 22 cooperate as sound source 33 for sound signal 36, i.e. means for generating a signal having an audible frequency component. In general, an audible frequency component has a frequency within the range from 20 Hz to 20 KHz. Signal DRV on line 34 is electromagnetic having an audible frequency component. Transducer 22 provides means for radiating these frequency components as sound.

Sound signal 38 is received by sensor 24 which converts sound energy into electromagnetic monitor signal MON on line 40. Circuit 42 receives signal MON on line 40, detects the modulation thereon, and applies the modulation to enhanced signal ENH on line 46. For manual monitoring purposes, signal ENH, drives speaker 26 to produce simulated speech sound signal 50 at conversational volume. Speech sound signal 50 in one embodiment includes audible frequency components that are out of phase with signals 36 and 38 to reduce the sound level of signals 36 and 38 outside the region local to sensor 24.

Control 52 includes electromechanical input devices such as switches, variable resistors, joy sticks, touch sensitive devices, and the like, for manual control inputs from the user. Manual control inputs allow the user to affect the intonation, volume, vibrato, reverberation, tremolo, randomization, attack, and decay functions well known in the music and speech simulator arts.

### SUMMARY OF THE INVENTION

This invention improves upon the prior art by extending implementations such as those described in U.S. Pat. No. 5,828,758. The invention broadly resides in a “digital audio larynx^{tm}” that processes the microphone's output coherently with the stimulus used to excite the vocal cavities. The use of coherent processing, implemented with a matched filter or a comb filter, allows complete removal of all of the stimulus from the recovered audio for a much cleaner reproduction.

The coherent processing is done in a digital signal processor (DSP) which is interfaced to an audio analog-to-digital (A/D) converter and other circuitry, including digital-to-analog converters (DACs). A microphone feeds the A/D, while the DACs feed amplifiers driving loudspeakers.

To facilitate hands-free operation, the microphone is mounted on a head-worn boom. One loudspeaker, also mounted on the boom alongside the microphone, is used to project the stimulus into the mouth. The other loudspeaker, contained within an enclosure along with batteries and other electronics, is used to broadcast the recovered speech. The enclosure is preferably small enough to fit in a shirt pocket or be worn on a lanyard or belt clip.

### BRIEF DESCRIPTION OF THE INVENTION

FIG. 1 is a block diagram of a prior art system; and

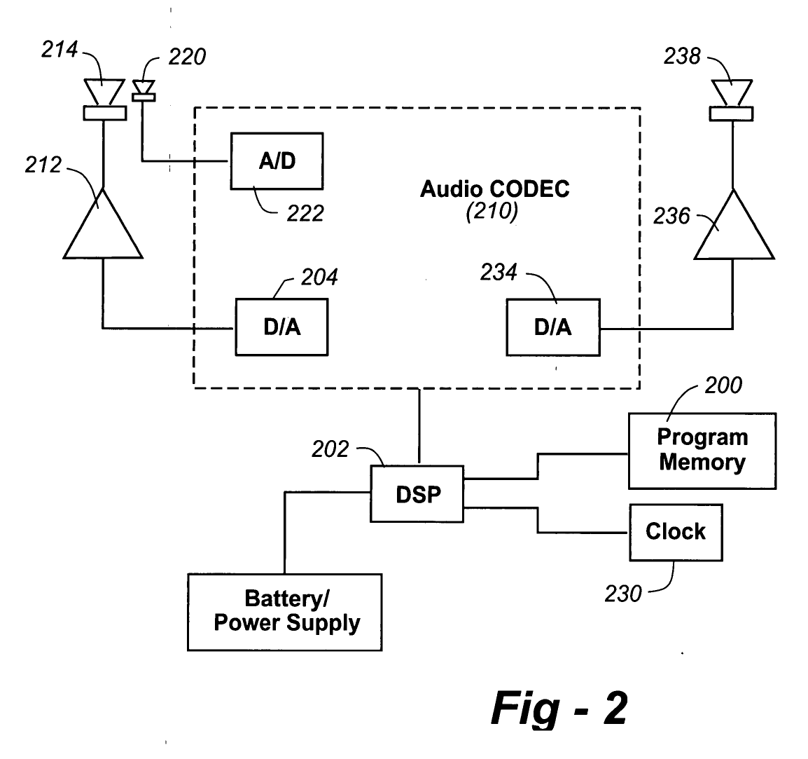

FIG. 2 is a block diagram of the preferred embodiment of the invention.

### DESCRIPTION OF THE INVENTION

As discussed in the Summary, this invention broadly resides in a prosthetic larynx that processes the input of a microphone coherently with the stimulus used to excite the vocal cavities. FIG. 2 is a block diagram of a preferred embodiment. The requisite stimulus is produced by a program stored in program memory 200 interfaced to a digital signal processor (DSP) 202. The various components are powered by supply 203.

The stimulus is produced as a sequence of digital numbers (samples) which are sent to the digital-to-analog converter (DAC) 204 in an audio coder-decoder (CODEC) 210. The DAC converters the sequence of numbers to a varying electrical signal which is amplified by amplifier 212 and sent to loudspeaker 214. The stimulus is preferably sub-audible.

Loudspeaker 214 is preferably mounted to a headset and projects its sound output into the mouth of the subject. A microphone 220 is positioned adjacent to loud speaker 214 on a headset boom, and recovers the sound from the subject's oral and nasal structure.

The output of the microphone 220 is sampled by analog-to-digital converter 222 in the CODEC 210, resulting in a sequence of numbers which are sent to the DSP 202. The sampling of the A/D and the D/A in the CODEC use a common clock 230, coherent processing of the microphone output is possible.

After processing the data stream from the microphone 220 to effectively remove the stimulus sent to loud speaker 214, DSP 2202 sends a stream of numbers representing the speech to a second DAC 234 within the CODEC 210. The second DAC 234 converters the number sequence to an electrical wave which is amplified at 236 and broadcast through loudspeaker 238.

The nature of the stimulus sent to loudspeaker 214 influences the performance of the system. For example, a burst of triangular waves at a frequency 50-300 Hz works well, though other waveforms are certainly possible. It has been found that rising and falling edges at different slopes gives greater modulated output.

As to the program in the DSP, assuming 24,000 samples per second and an 80 Hz fundamental stimulus, the stimulus will be a pattern stored as 300 samples and output sequentially. A comb filter is implemented as a circular buffer of 300 samples, and a simple subtraction of the oldest sample from the newest being the output of the coherent comb filter. The comb filter's output is further processed by additional filters to reduce the acoustic feedback that such systems can produce. Matched filtering may alternatively be used.

1. A method of generating artificial speech, comprising the steps of:
providing a stimulus into the mouth of a subject;
recovering sound resulting from the stimulus as modified by the subject's oral and nasal structure;
converting the recovered sound into an electrical signal; and
coherently processing the electrical signal to effectively remove the stimulus.

2. The method of claim 1, further including the step of converting the coherently processed electrical signal into an audible signal.

3. The method of claim 1, wherein the stimulus is generated by a programmed digital signal processor.

4. The method of claim 1, wherein the stimulus is a burst of triangular waves at a frequency in the range of 50-300 Hz.

5. The method of claim 1, wherein the stimulus is a burst of triangular waves with and rising and falling edges at different slopes.

6. The method of claim 1, wherein the step of coherently processing the electrical signal involves the use of a comb filter.

7. The method of claim 1, wherein the step of coherently processing the electrical signal involves the use of a matched filter.

8. A system for generating artificial speech, comprising:
circuitry, including a first loudspeaker, for providing a stimulus into the mouth of a subject;
a microphone for recovering sound resulting from the stimulus as modified by the subject's oral and nasal structure;
circuitry for converting the recovered sound into an electrical signal; and
a processor for coherently processing the electrical signal to effectively remove the stimulus.

9. The system of claim 8, further including a second loudspeaker for converting the coherently processed electrical signal into an audible signal.

10. The system of claim 8, wherein the processor is a digital signal processor.

11. The system of claim 8, wherein the stimulus is a burst of triangular waves at a frequency in the range of 80-100 Hz.

12. The system of claim 8, wherein the stimulus is a burst of triangular waves with and rising and falling edges at different slopes.

13. The system of claim 8, wherein the processor further includes a comb filter.

14. The system of claim 8, wherein the processor further includes a matched filter.

15. The system of claim 8, wherein the microphone and first loudspeaker are supported on a head-mounted boom for hand's free operation.
